(19) United States
(12) Patent Application Publication
(10) Pub. No.: US 2020/0363360 A1
(43) Pub. Date: Nov. 19, 2020

(54) DIRECT CURRENT DRIVE CIRCUITRY DEVICES

(71) Applicant: Hewlett-Packard Development Company, L.P., Spring, TX (US)

(72) Inventors: Matthew David SMITH, Corvallis, OR (US); Rachel M. WHITE, Corvallis, OR (US)

(73) Assignee: Hewlett-Packard Development Company, L.P., Spring, TX (US)

(21) Appl. No.: 16/766,163

(22) PCT Filed: Jan. 23, 2018
(86) PCT No.: PCT/US2018/014785

§ 371 Date: May 21, 2020

(51) Int. Cl. G01N 27/04; G01N 33/49; G01N 27/416; G01R 19/257

(52) U.S. Cl. CPC G01N 27/045; G01R 19/257; G01N 27/4161; G01N 33/49

(57) ABSTRACT

A direct current drive circuitry device can include a pull-up resistor to receive an input voltage and an electrical interface positioned in series and downstream from the pull-up resistor. The electrical interface can be electrically coupleable to a grounded microfluidic sensor to form a voltage divider circuit in combination with the pull-up resistor to generate an output voltage at the voltage divider circuit. The circuit can include an electrical switch to receive and charge cycle (discharging period and a charging period) the input voltage to the pull-up resistor of the voltage divider circuit. An analog-to-digital convertor can be electrically coupled to the voltage divider circuit (once completed) to measure the output voltage. A voltage buffer amplifier can be positioned between the voltage divider circuit and the analog-to-digital converter to prevent the analog-to-digital converter from loading the voltage divider circuit.

### BACKGROUND

Biological testing, such as the testing of blood or nucleic acids for various properties, can be carried out for purposes of diagnostics and/or treatment. For example, blood drawn from patients taking oral anticoagulants in response to a medical condition (e.g. stroke, pulmonary embolism, etc.) can be tested for coagulation properties. Coagulation testing can also be performed for clinical diagnostic purposes. Blood cell counting can also be carried out to help diagnose any of a number of medical conditions, or to provide medical information regarding the condition of blood resulting from medical treatment. In other examples, nucleic acid testing (NAT) or a nucleic acid amplification testing (NAAT) can also be used for diagnostics, such as when testing for pathogens in a specimen of blood or other bodily fluid.

### BRIEF DESCRIPTION OF THE DRAWINGS

Additional features and advantages of the disclosure will be apparent from the detailed description which follows, taken in conjunction with the accompanying drawings, which together illustrate, by way of example, features of the present technology.

FIG. 1 is a diagram of an example direct current drive circuitry device in accordance with examples of the present disclosure;

FIG. 2 is a diagram of an example direct current drive circuitry device, which can be electrically coupled to an example microfluidic chip with a microfluidic sensor, in accordance with examples of the present disclosure;

FIG. 3 depicts an example charge cycle at a voltage divider circuit, wherein the voltage divider circuit is provided by a microfluidic sensor connected in series downstream from a pull-up resistor of a direct current drive circuitry device in accordance with examples of the present disclosure;

FIG. 4 is a diagram of an example direct current drive circuitry device with an added low impedance rapid discharge pathway, which can be electrically coupled to an example microfluidic chip with a microfluidic sensor, in accordance with examples of the present disclosure; and

FIG. 5 is a diagram of an example direct current drive circuitry device with an added low impedance rapid discharge pathway, which can be electrically coupled to an example microfluidic chip with a microfluidic sensor, in accordance with examples of the present disclosure

Reference will now be made to several examples that are illustrated herein, and specific language will be used herein to describe the same. It will nevertheless be understood that no limitation of the scope of the disclosure is thereby intended.

### DETAILED DESCRIPTION

Many biological fluids can be tested or otherwise sensed using microfluidic sensors that interact with the biological fluid electrically. For example, the sensing of an electrical property or magnitude of an electrical property, or the sensing of changes in an electrical property or magnitude of an electrical property, e.g., resistance, capacitance, etc., in the presence of a biological fluid can provide information that can be correlated to a biological property or biological response, e.g., blood coagulation, the presence of a pathogen infecting nucleic acids within a biological fluid, the concentration of cells in a biological flood, etc. Thus, electrically sensed measurements of various types can provide valuable information about the biological fluid being tested. In other examples, rather than providing electrically sensed biological fluid properties per se that correlate more directly with the assayed biological fluid, e.g., coagulation, cell counting, nucleic acid properties, etc., some more independent biological fluid properties can also be sensed or “observed” for measurements taken at a microfluidic sensor location, or at an off-sensor (or off-chip) location. An example of this may be the measurement of temperature, such as by the use of a thermal sensor resistor (TSR). In this case, the TSR may act as a “microfluidic sensor” of sorts, where resistance of the TSR changes as a function of biological fluid temperature. In this later case, logic inside of off-sensor or off-chip drive electronics can be used in conjunction with a microfluidic sensor (or TSR in this instance) to correlate the TSR resistance with actual temperature values. Regardless of the purpose or structure of the microfluidic sensor (or microfluidic chip which includes a microfluidic sensor), there are architectures suitable for testing various types of biological fluids. As a note, the term “electrical sensor” is used broadly to include electrical devices that contact a sample fluid that can be used to determine any property of the sample fluid, whether biological or non-biological. Examples of electrical sensors can thus include electrical sensors per se, TSRs (described above), electro-chemical sensors or devices, electro-optical sensors or devices, electro-mechanical sensors or devices, or a combination thereof. Furthermore, whether the microfluidic sensors described herein are adapted for testing or sensing a biological property, sensing a non-biological property, e.g., temperature, or otherwise electrically interact with a biological fluid in another manner, the term “microfluidic sensor” can be used due to its information gathering properties or even information gathering assistance provided with respect to the biological fluid. In further detail, the term “microfluidic chip” can be used to describe a microchip device that can include a microfluidic sensor, whether used for electrically testing or sensing a biological fluid or some other type of fluid sample.

With this in mind, the present disclosure is drawn to a direct current drive circuitry device, such as that suitable for use with microfluidic sensors, including microfluidic sensors present on microfluidic chips. In other examples, in combination, the direct current drive circuitry device and the microfluidic sensor can be assembled or electrically coupled together to provide direct current electrical sensing systems.

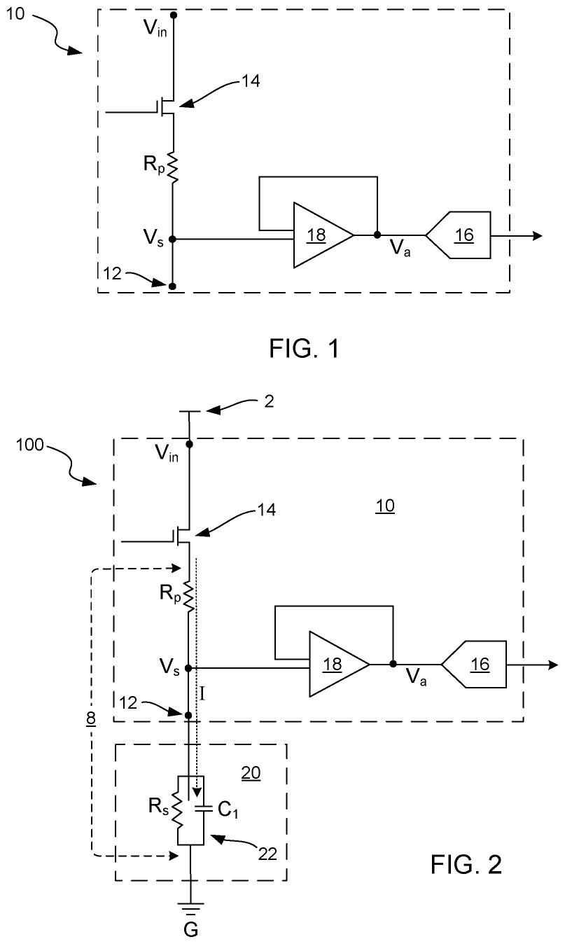

In FIG. 1, a direct current drive circuitry device 10 is shown that can be coupled to a voltage source (not shown, but shown in FIG. 2) at an input voltage (Vᵢₙ) location. The direct current drive circuitry device can also be couplable to a microfluidic sensor (not shown, but shown in FIG. 2) at electrical interface 12, positioned in series and downstream with respect to a pull-up resistor, shown at resistance (Rₚ). The direct current drive circuitry device can also include an electrical switch 14 to receive and charge cycle the input voltage received from the voltage source to and through the pull-up resistor, forming a voltage divider circuit with the microfluidic sensor once connected, as described in greater detail hereinafter. An output voltage, or sensed voltage (Vₛ), can be measured as a voltage drop across the voltage divider circuit using an analog-to-digital converter 16. In further detail, a voltage buffer amplifier 18 can be electrically connected at the voltage divider circuit (located between the pull-up resistor and the microfluidic sensor shown in FIG. 2). The voltage buffer amplifier can, for example, be a unity gain buffer amplifier having a voltage gain where Vₐ is about 1, thereby providing the output voltage to analog-to-digital converter at a voltage level that is about equivalent to output voltage prior to passing through the unity gain buffer amplifier. In some examples, amplification can be greater than 1, where Vₐ is greater than 1.

In further detail, as shown in FIG. 2, a direct current drive circuitry device 10 similar to that shown in FIG. 1 is shown as part of a direct current electrical sensing system 100. The direct current drive circuitry device can include a pull-up resistor, shown at resistance (Rₚ) and an electrical interface 12 positioned in series and downstream from the pull-up resistor. The direction of the current (I) during charging periods is shown schematically by a dotted arrow. Discharging can occur as charge built up on the microfluidic sensor 22 goes to ground, shown at (G). As a note, though a pathway to ground is shown simply as a direct pathway beneath the microfluidic sensor in FIG. 2, it is appreciated that ground pathways can be found anywhere where grounding is used. For example, the ground pathway from the microfluidic sensor could be routed anywhere within the system, including back onto a chip or device that may carry the direct current drive circuitry, for example. Furthermore, there can also be other examples where more rapid discharging periods can occur using a rapid discharge pathway to ground, which is shown and described in greater detail in FIG. 4.

Also shown in FIG. 2, the electrical interface 12 can be electrically coupleable to a grounded microfluidic sensor 22 of a microfluidic chip 20 to form a voltage divider circuit 8. In this example, the microfluidic sensor is shown as coupled to the electrical interface. The voltage divider circuit can include a combination of the pull-up resistor (shown at resistance (Rₚ), and the microfluidic sensor. The direct current drive circuitry device 10 can also include an electrical switch 14 to receive and charge cycle an input voltage (Vᵢₙ) from a voltage source 2 to the pull-up resistor of the voltage divider circuit, e.g., one charge cycle can include a discharging period and a charging period. Within a charge cycle, in some examples, a sampling cycle (which can be defined to include a sampling cycle rate measured typically in microseconds to indicate a rate at which measurements may be taken) can be once per charge cycle. However, in other examples, multiple sampling cycles can occur during a single charge cycle, depending on the application. The pull-up resistor, for example, can help to modulate a voltage drop that can occur across the voltage divider circuit. An analog-to-digital convertor 16 can be electrically coupled to the voltage divider circuit to measure an output voltage, or sensed voltage (Vₛ), which can include a sensed voltage drop across the voltage divider circuit. Upon charging up the voltage divider circuit, the output voltage can be measured, in this example, at the analog-to-digital converter and then the electrical switch can be turned to an OFF position to initiate a discharging (or charge interruption) period at the voltage divider circuit. In further detail, a voltage buffer amplifier 18 can be positioned between the voltage divider circuit and the analog-to-digital converter to prevent the analog-to-digital converter from loading the voltage divider circuit. In one specific example, the input voltage can be from 0.1 V to 5 V. In another example, the pull-up resistor can have a resistance from 10 KOhm to 2 MOhm. Sub-ranges of these voltages and resistances, or even voltages and/or resistances outside of these ranges, can likewise be used in some instances. The analog-to-digital converter can convert the output voltage (after passing through the voltage buffer amplifier) to a digital output value related to a magnitude of the output voltage.

The digital output value can have a resolution of 0.02 V or less per digital output value change, in one example. The voltage buffer amplifier can, for example, be a unity gain buffer amplifier having a voltage gain of about 1 (e.g., A=1), thereby providing the output voltage to analog-to-digital converter at a voltage level that is about equivalent to output voltage prior to passing through the unity gain buffer amplifier. Examples of unity gain buffer amplifiers that can be used include an operational amplifier or a common drain amplifier. In one specific example, if the voltage buffer amplifier can be greater than about 1, then the amplifier can generate a voltage gain (Vₐ), but can still operate as a voltage buffer amplifier to avoid loading the voltage divider circuit (when connected to a microfluidic sensor).

In another example, also illustrated by example in FIG. 2, a direct current electrical sensing system, shown generally and collectively at 100, can include a voltage source 2, the direct current drive circuitry device 10, and the microfluidic sensor 20. The voltage source can generate an input voltage (Vᵢₙ) suitable for driving the direct current drive circuitry of the device, but in one example, can range from 0.1 V to 5 V. The direct current drive circuitry device can include an electrical switch 14 to charge cycle the input voltage (from the voltage source) between charging periods and discharging periods. A voltage divider circuit 8 can be included to receive the input voltage charge cycled by the electrical switch. The voltage divider circuit can generate an output voltage, or sensed voltage (Vₛ) drop across the voltage divider circuit that is lower than the input voltage, e.g., a fraction of the input voltage can be sensed or generated when distributed between two devices or components of the system. The voltage divider circuit can include a pull-up resistor, shown at resistance (Rₚ), having a resistance from 1 KOhm to 2 MOhm, as well as a microfluidic sensor 22 that is grounded, and which is connected downstream and in series with the pull-up resistor. Notably, the pull-up resistor can be part of the direct current drive circuitry of the device, and the microfluidic sensor can be part of a separate microfluidic chip 22. Thus, when the drive circuitry is electrically coupled to the microfluidic chip (via an electrical interface 12, for example), the voltage divider circuitry is in place to receive charging voltages from the voltage source and the electrical switch, and can discharge when switched to an OFF position using any of a number of types of switching circuitry. In one example, the microfluidic sensor can include a sensor resistor, shown at resistance (Rₛ), as well as other possible electrical component(s), e.g., capacitors (shown by example at C₁), diodes, light-emitting diodes (LEDs), transistors, integrated circuits, etc. Thus, in the specific example shown in FIG. 2, the microfluidic sensor is modeled using a resistor and capacitor in parallel, but many other circuitry arrangements can be used for microfluidic sensors. In further detail in this example, an analog-to-digital convertor 16 can be electrically coupled to the voltage divider circuit to measure the output voltage. A voltage buffer amplifier 18 can also be positioned between the voltage divider circuit and the analog-to-digital circuit to prevent the analog-to-digital converter from loading the voltage divider circuit. In one example, the analog-to-digital converter can convert the output voltage to a digital output value related to a magnitude of the output voltage, and the digital output value can have a resolution of 0.02 V or less per digital output value change. In another example, the voltage buffer amplifier can be a unity gain buffer amplifier having a voltage gain of about 1 (e.g., A=1). Example unity gain buffers can include operational amplifiers or common drain amplifiers. This arrangement can deliver the output voltage to the analog-to-digital converter in a manner that is about equivalent to the output voltage, or sensed voltage (Vₛ), prior to passing through the unity gain buffer amplifier. In another example, however, the voltage buffer amplifier can generate a voltage gain from about 1 to about 100, thereby providing a voltage (Vₐ) measurement at the analog-to-digital converter that is amplified to include a voltage gain compared to the output voltage prior to passing through the voltage buffer amplifier. If amplifying the voltage in this manner, a lower input voltage can be used in some examples, e.g., from 0.1 V to 2 V. As a note, when using a voltage buffer amplifier that is a unity buffer amplifier, the output or sensed voltage (Vₛ) can be equal to the amplified voltage, and thus, output voltage gain levels, shown at Vₐ, are not particularly relevant, as output or sensed voltage (Vₛ) would be about equal to the “amplified” voltage (Vₐ), e.g., (A=1). In still another example, the sensor resistor (Rₛ) of the microfluidic sensor can have a resistance within one order of magnitude of the pull-up resistor, e.g., if the pull-up resistor is 1 MOhm then the sensor resistor can be 100 KOhms or more up to about 1 MOhm. In one example, the pull-up resistor can have a resistance that is less than or about equal to the sensed value. In further detail, the microfluidic sensor can be part of a microfluidic chip or device that includes a microfluidic testing chamber suitable for receiving a biological fluid which contacts the microfluidic sensor.

In another example, also as shown in FIG. 2, a direct current electrical sensing system 100 can include a voltage source 2 to generate an input voltage (Vₙ) ranging from 0.1 V to 5 V and an electrical switch 14 to charge cycle the input voltage between a charging state and an interruption state. A voltage divider circuit 8 can also be present to receive the input voltage charge cycled by the electrical switch. The terms “charge cycle” or “charging cycle” can include both a discharging period and a charging period at the voltage divider circuit. Thus, an “input voltage” can be charged cycled by turning an electrical switch OFF and ON to cause a discharging period and a charging period, for example. The voltage divider circuit can generate an output voltage, or sensed voltage (Vₛ), that is lower than the input voltage, and can include a pull-up resistor, shown at resistance (Rₚ), which is shown as part of direct current drive circuitry device 10, and a microfluidic sensor 22, which is shown as part of microfluidic chip 20. The pull-up resistor can have a resistance, on one example, from 1 KOhm to 2 MOhm. The microfluidic sensor can be connected downstream and in series with the pull-up resistor. An analog-to-digital convertor 16 can be electrically coupled to the voltage divider circuit to measure the output voltage, and a voltage buffer amplifier 18 can be positioned between the voltage divider circuit and the analog-to-digital convertor to prevent the analog-to-digital convertor from loading the voltage divider circuit.

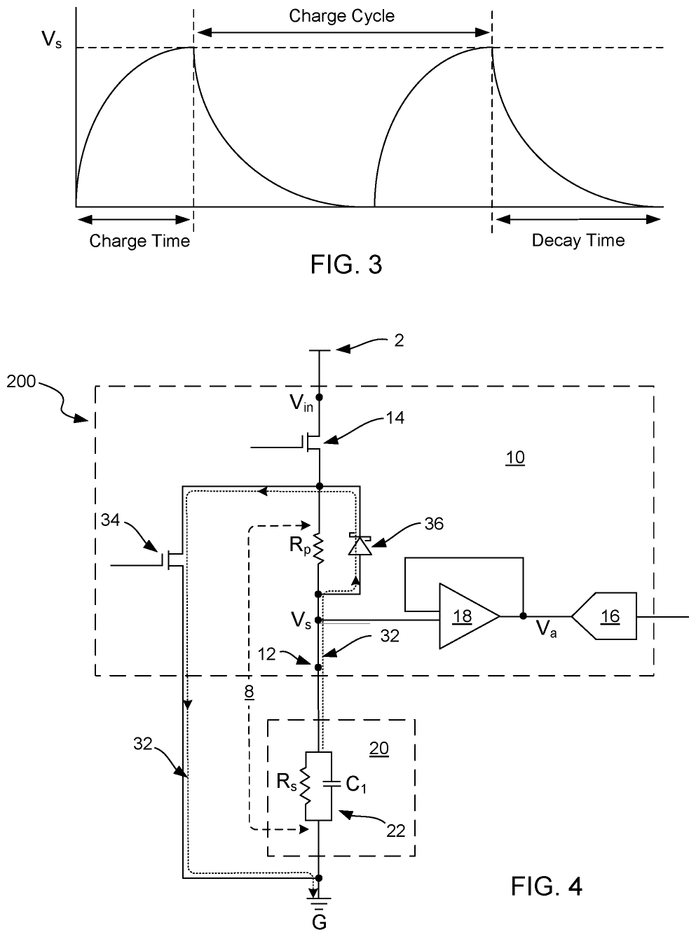

In further detail and as also shown in FIG. 3, the output voltage, or sensed voltage (Vₛ), can be measurable within one charge cycle at a sampling cycle rate from 1 microsecond to 1000 microseconds, for example. To illustrate, the output voltage can be measurable at the analog-to-digital converter upon charging the voltage divider circuit, and the output voltage can then be again measurable at the analog-to-digital converter after one charge cycle which includes one discharging period and one charging period. Thus, during a charge cycle, one or more measurements can be taken at a sampling cycle rate from 1 microsecond to 1000 microseconds (which can coincide in time with the charge cycle, or can shorter or longer than the charge cycle). For example, it can be possible to take multiple measurements during a charge cycle, such as during the charging period, to give more information about the state of the fluid. The microfluidic chip 20, which can include a microfluidic sensor 22 with a sensor resistor, shown at resistance (Rₛ), can be designed for contact with a sample fluid, such as a biological fluid. In one specific example, a diluted blood sample can be the biological fluid and can be diluted for a cell counting application at a sampling cycle rate from 10 microseconds to 100 microseconds, e.g., counting blood cells by measuring or detecting disturbances in the electric field. In another specific example, a blood sample for evaluating coagulation properties can be carried out a sampling cycle rate from 100 microseconds to 350 microseconds, e.g., determining blood coagulation through the change in resistance of a blood sample resulting from time passage, introduction of heat, introduction of a chemical agent that interacts with the blood, etc. In yet another specific example, a nucleic acid sample can be evaluated, such as for the presence of a pathogen, at a sampling cycle rate from 1 microsecond to 1000 milliseconds. Other biological tests and/or other sampling rates can likewise be conducted or used with these systems.

In further detail, and as shown by way of example in FIGS. 2 and 3, the electrical switch 14 can control application of the input voltage to the system (from a voltage source 2, for example), but this may not occur as a perfect square wave. Typically, there can be some delay between when input voltage is applied and when the output voltage, used as a sensed voltage (Vₛ), reaches at or near its maximum charge for measurement at the analog-to-digital converter 16 which can be isolated by the voltage buffer amplifier 18. There can be a time interval between when the output or sensed voltage is measured by the analog-to-digital converter, and when the next measurement can occur, e.g., upon measurement the voltage divider circuit can be discharged and charged again for its next peak (or near peak) measurement to occur. Thus, this time interval can collectively include a decay time followed by a charge time. In other words, when voltage divider circuit is at or near its measuring charge (peak or near peak), the system can take a measurement and the input voltage can be switched off, thereby discharging (shown as “decay time”) the system followed by a charging (shown as “charge time”).

Measurements can be taken when the voltage divider circuit is charged, typically at or near a charge peak, but can also be taken at other states of charge at the voltage circuit divider. In relation to a charge cycle as previously defined, a “sampling cycle” (which can be defined to include a “sampling cycle rate”) can correspond to one full charge cycle (e.g., from peak charge to discharge to another peak charge), or can be longer than a charge cycle (e.g., such as when waiting to take a measurement after a full charge is reached), or can be shorter than a full charge cycle (e.g., such as for taking multiple measurements during a charge cycle). Thus, the sampling cycle rate can refer to time interval used to take measurements during a charge cycle. Thus, a shorter time interval indicates a quicker sampling cycle rate. For example, one sampling cycle can overlap with both a discharging period and a charging period of a charge cycle. Within a charge cycle, in some examples, a sampling cycle can be once per charge cycle, e.g., at full charge a measurement is taken, followed by discharging and charging to take another measurement. However, in some examples, a sampling cycle can have a shorter sampling cycle rate compared to the charge cycle, and thus, samples can be measured multiple times during a single charge cycle, depending on the application. Thus, the charge time can be controlled by a user, or can be automatically determined using software or other controllers, or can be controlled by other electrical components or circuits that may also be present on the direct current drive circuitry device 10. When evaluating charge time for circuitry or system design, for example, shortening the charge time can increase the measuring frequency, but can in some instances limit sensitivity of the system for some types of biological phenomena. For example, there may be some biological pathways that would benefit from more recovery time between charging periods and measurements. On the other hand, increasing the charge time can increase signal strength that is measured, but in some instances can adversely affect the sample itself, and/or can lead to electrolysis. Thus, the cycling time, or sampling rate, within an acceptable range can be specific for a given biological fluid sample to achieve desired results. Without being limiting, as an example, some biological fluids can benefit from a sampling cycle rate ranging from 1 microsecond to 1000 microseconds, from 1 microsecond to 350 microseconds, from 350 microseconds to 1000 microseconds, from 10 microseconds to 350 microseconds, from 10 microseconds to 100 microseconds, from 100 microseconds to 350 microseconds, from 25 microseconds to 300 microseconds, from 50 microseconds to 250 microseconds, from 50 microseconds to 200 microseconds, etc.

In more specific detail regarding various input voltages and resistances across the voltage divider circuit, the input voltage can typically range from about 0.1 V to about 5 V for applications where a unity buffer amplifier is used, or the input voltage can be from about 0.1 V to about 2 V for applications where a voltage buffer amplifier is used that generates a voltage gain. However, in other examples, the input voltage can be from 0.5 V to 5 V from 1 V to 4 V from 2 V to 4 V from 3 V to 4 V from 3 V to 3.5 V from 0.5 V to 2 V from 0.5 V to 1.5 V etc., particularly when using a unity gain amplifier with a voltage gain of about 1. The resistance at the pull-up resistor can be, in some examples, from about 10 KOhm to 2 MOhm. Likewise, the resistance at the pull-up resistor can alternatively be from 10 KOhm to 1 MOhm, from 10 KOhm to 500 KOhm, from 50 KOhm to 1.5 MOhm, from 100 KOhm to 1 MOhm, from 200 KOhm to 1 MOhm, from 100 KOhm to 750 KOhm, from 100 KOhm to 500 KOhm, from 1 MOhm to 2 MOhm, from 500 KOhm to 1.5 MOhm, etc. Other resistances can also be used outside of these ranges, depending on the specific application, system architecture, biological fluid being sampled, etc. For example, the resistor can be an adjustable resistor, e.g., a potentiometer or a combination of multiple resistors that could be switched into or out of the circuit. In further detail, the circuit can include an auto-range or auto-adjustment feature that can be adjusted automatically. Auto-range or auto-adjustment features can be triggered or programmed to operate in a certain manner depending on the type of fluid being measured, the result of previous measurement(s), etc. With respect to the microfluidic sensors which include a sensor resistor, the sensor resistor can be within one order of magnitude of the pull-up resistor. For example, a ratio of pull-up resistor resistance (Rₚ) to sensor resistance (Rₛ) can be from 1:10 to 10:1, from 1:10 to 2:1, from 1:2 to 10:1, from 1:10 to 1:1; from 1:1 to 10:1, from 1:5 to 5:1, from 1:2 to 2:1, from 2:1 to 10:1, from 2:1 to 5:1, etc. Thus, in many examples, the pull-up resistor can have a resistance (Rₚ) within an order of magnitude higher than the resistance (Rₛ) of the sensor resistor. However, there are examples where the pull-up resistor can have a resistance that is lower than the resistance at the microfluidic sensor as well, as reflected in the ranges set forth above. In other examples, irrespective of these resistance ratios, the sensor resistor can have a resistance ranging from 10 KOhm to 1 MOhm, from 10 KOhm to 500 KOhm, from 50 KOhm to 1.5 MOhm, from 100 KOhm to 1 MOhm, from 200 KOhm to 1 MOhm, from 100 KOhm to 750 KOhm, from 100 KOhm to 500 KOhm, from 1 MOhm to 2 MOhm, from 500 KOhm to 1.5 MOhm, etc.

In further detail regarding the sensor resistor, shown at resistance (Rₛ) of the microfluidic sensor 22, resistance can be inferred by equation (1) as follows:

Rₛ=Rₚ(Vₛ)/Vᵢₙ-V_{s (}1)

where Rₛ is the (unknown) resistance at the sensor resistor, Rₚ is the (known) resistance at the pull-up resistor, Vₛ is the (measured) output voltage, and Vᵢₙ is the (known) input voltage. From this equation, Rₛ can be determined, which can provide electrical information that correlates to a biological fluid property, for example. As an example, if Vᵢₙ is 3.3 V Vₛ is 1 V and Rₚ is 1 MOhm, then Rs is about 435 KOhm. This resistance value can provide information about the biological fluid that is in contact with the microfluidic sensor, for example.

Turning now to FIG. 4, a direct current drive circuitry device 10 is shown that is similar to that shown in FIG. 2, but which further includes an additional low impedance rapid discharge pathway 32, which is used in conjunction with two electrical switches 14, 34 that are essentially oppositionally timed (e.g., respectively ON/OFF during charging periods and OFF/ON during discharging periods) during the charge cycle. The “oppositional” timing can be any ratio of discharging and charging time that is practical for a given application, e.g., about 50:50, 60:40, 40:60, 70:30, 30:70, 60:40 to 40:60, 70:30 to 30:70, etc. There can also be periods where both electrical switches are in an OFF position. Furthermore, as there can be some delay inherent in opening and closing electrical switches, some overlapping OFF time may occur due to this delay, or on the other hand, this delay can be accounted for to generate as much charging and discharging time possible during a rapid charge cycle. In further detail, regardless of ON and OFF timing of the two respective electrical switches, the discharging time, or “decay time” shown in FIG. 3, can be reduced further, allowing for more rapid cycling of direct current electrical sensing systems 200. This can provide, in some examples, faster sampling or measurement rates, e.g., one measurement per charge cycle, two measurements per charge cycle, three measurements per charge cycle, etc. Furthermore, in this example, like FIG. 2, the direct current drive circuitry device can include a pull-up resistor, shown at resistance (Rₚ), and an electrical interface 12 positioned in series and downstream from the pull-up resistor. The electrical interface can be electrically coupleable (or coupled) to a grounded microfluidic sensor 22 of a microfluidic chip 20 to form a voltage divider circuit 8. The voltage divider circuit can include a combination of the pull-up resistor and the microfluidic sensor. The direct current drive circuitry device, as with FIG. 2, can also include a voltage buffer amplifier and an analog-to-digital converter, as previously described. As a note, in FIG. 4, the charging pathway is not shown specifically, but can be the same as that shown at current (I) shown in FIG. 2. Rather, in FIG. 4, only the rapid discharge pathway 32 is shown so as to not obscure this example showing this particular pathway detail.

In further detail regarding FIG. 4, the direct current drive circuitry device can also include an electrical switch 14 to receive and charge cycle (ON and OFF) an input voltage (Vᵢₙ) from a voltage source 2 to the pull-up resistor, shown at Rₛ, to ultimately charge a voltage divider circuit 8, e.g., electrical switch can be ON during charging periods and OFF during discharging periods. The electrical switch in this example can be referred to as a charging electrical switch, as it puts the voltage divider circuit into a charging period when the charging electrical switch is in the ON position, e.g., allowing current to flow downstream to the microfluidic sensor 22. However, it is noted that during charging, a second electrical switch 34 (which can be referred to as a discharging electrical switch) can also be included). The second (discharging) electrical switch can be present along a low impedance rapid discharge pathway 32. Thus, while the (charging) electrical switch 14 is in an ON position, the second (discharging) electrical switch 34 can be in an OFF position. In the OFF position, charging of the voltage divider circuit can occur as the second (discharging) electrical switch prevents the current from going to ground along the rapid discharge pathway, and normal charging of the voltage divider circuit can occur. Notably, the rapid discharge pathway includes a Schottky diode 36 in this example, which can provide two benefits. First, the Schottky diode can prevent current from bypassing the pull-up resistor, shown at resistance (Rₚ), during charging periods, as the pull-up resistor is part of the voltage divider circuit that is charged during the charging periods. The Schottky diode does not allow current to flow backward, e.g., it is essentially unidirectional. Second, during discharging periods, where the (charging) electrical switch 14 is in the OFF position and the second (discharging) electrical switch is in the ON position, the Schottky diode allows for essentially free movement of current along the rapid discharge pathway in the direction shown in FIG. 4 by the Schottky diode symbol, e.g., discharge can occur from the charged capacitor C1 through the Schottky diode (rather than the pull-up resistor) and further through the second (discharging) electrical switch which is in the ON position leading to ground. The electrical (charging) switch during discharging periods can be in the OFF position. This rapid discharge pathway, thus, allows for faster discharge of the voltage divider circuit during discharging periods because of the relative low impedance that can be present along this pathway compared to the relative high impedance that may be present when more directly discharging the microfluidic chip or sensor to ground. To be clear, some discharge can occur along a higher impedance grounding pathway (shown below the microfluidic sensor 22), but in this example, there may be a shorter discharging period by providing a second relative low impedance rapid discharge pathway, such as that shown in FIG. 4 at pathway 32.

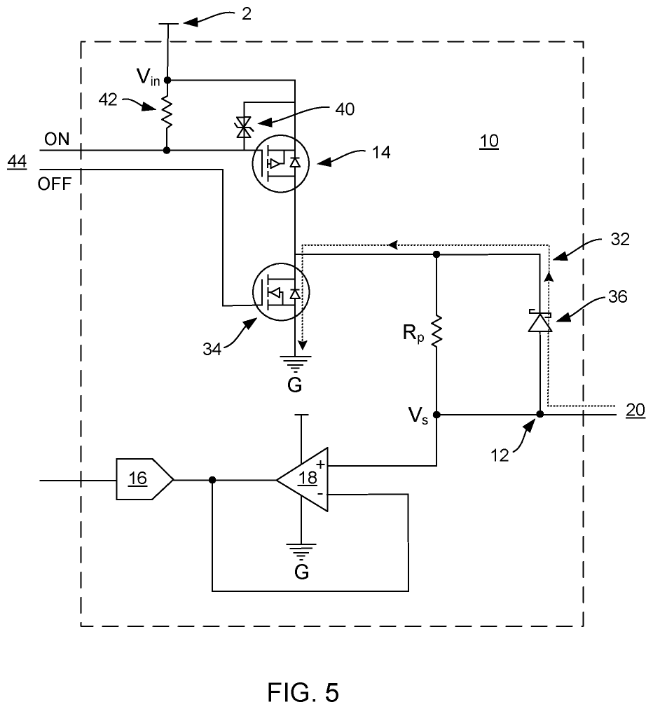

FIG. 5 depicts an alternative example including some of the same features shown in FIGS. 1 and 2, and in further detail in FIG. 4. Some of these features can include various connection locations shown at input voltage (Vᵢₙ) for connecting the voltage source 2 as well as electrical interface 12 for connecting the microfluidic sensor 20. Other similar features can include a pull-up resistor shown at (Rₚ), an output or sensed voltage (Vₛ) connection or area, an analog-to-digital converter 16, a voltage buffer amplifier 18, rapid discharge pathway 32 including a Schottky diode 36 positioned in parallel with the pull-up resistor as previously described, alternating electrical switches 14, 34 which in this example can be field effect transistors (FETs). FETs are unipolar transistors that can use electric field to control the shape/electrical conductivity of a switch channel within a semiconductor material. In further detail, there are other example features shown in FIG. 5 that are not shown or described in FIGS. 1, 2, and 4. For example, FIG. 5 includes a transient voltage suppression diode (TVS), or thyrector, which can be used to protect the electronics from voltage spikes that may be induced from the connected wires. An additional resistor 42 is also shown to resist current flow back to the voltage source when current is flowing along the ON pathway shown at 44 (either off-chip or to another component that may be on the chip). An OFF pathway is also shown. Thus, in one example, it can be up to the chip driving these two lines to make them inverts of one another. Multiple ground locations (G) are also shown, and notably, in this example, the rapid discharge pathway of the microfluidic sensor is shown being routed back onto the direct current drive circuitry device.

Regarding the direct current drive circuitry device components or integrated circuits that can be used, further detail is provided for example purposes only, and various other variations of these examples, or even other components or integrated circuits, can be implemented with the direct current drive circuitry device shown generally at 10 in FIGS. 1, 2, 4, and 5, e.g., circuitry to control cycling of the electrical switch 14, circuitry for collection and storage collected data, circuitry to process data, circuitry to send data to a computing device or to another microchip for processing or display, etc. In other words, the drive circuitry is shown in a simplified manner so as not to obscure the present disclosure.

The direct current drive circuitry device 10 can include an electrical switch 14 to essentially turn ON and OFF the direct current received from either an on-chip or off-chip voltage source 2. The switch can be any electrical component, such as a switch, a gate, a transistor, or any other component that can be “opened” and “closed” to rapidly allow the voltage divider circuit 8 to become charged and discharged in a cyclical manner suitable for generating useable output voltages. Full charging and full discharge can be beneficial in some examples, but in other examples, partial charging and/or partial discharging can occur. Thus, the electrical “switch” may or may not provide total cutoff of voltage when in a relative “OFF” position. Examples of electrical switches can include BJTs, NPN transistors, PNP transistors, relays, etc.

The direct current drive circuitry device 10 can also include a pull-up resistor, shown at resistance (Rₚ), which can help modulate a voltage drop that can occur across the voltage divider circuit 8. For example, when the switch 14 physically interrupts the connection between a voltage source 2 and a ground associated with the microfluidic sensor 22, the pull-up resistor can provide for maintaining a well-defined voltage across the microfluidic sensor 22 during voltage interruption or discharging periods.

In further detail, the direct current drive circuitry device 10 can include analog-to-digital converter 16 to convert the output or sensed voltage (Vₛ), or in some cases an output voltage after a voltage gain (Vₐ), to a digital value. The digital value can provide a voltage sensitivity that is related to the resolution of the analog-to-digital converter. As mentioned, a digital output value can have a resolution of 0.02 V or less per digital output value change. In other examples, the resolution can be 0.002 V or less, or 0.00122 V or less, per digital output value change. In further detail, the resolution can vary depending on the resolution of the analog-to-digital converter, e.g., 8 bit, 10 bit, 12 bit, etc., and the reference voltage of the analog-to-digital converter, e.g., 2.5V 3.3V, 5V etc. As a specific example, the 0.00122 V of one of the ranges listed above, e.g., 0.00122 V or less, comes from a 5V reference and a 12 bit analog-to-digital converter, meaning that each 0.00122V change (5/2¹²=0.00122) causes the analog-to-digital converter to change the digital output value by 1 count. The resolution can also be defined electrically as the minimum change in voltage (AV) to cause a change in output code, which is sometimes referred to as the least significant bit (LSB) voltage. In other words, the resolution can be equal to the LSB voltage. Thus, voltage can be converted to a digital number or other value that is associated with a magnitude of the voltage. This can be a “two's complement” binary number that is proportional to the input or can be some other value that provides similar magnitude digital information. In some examples, the analog-to-digital converter can be or can include an integrated circuit (IC).

The direct current drive circuitry device 10 can also include a voltage buffer amplifier 18. In this example, when transferring the output voltage, or sensed voltage (Vₛ), from the voltage divider circuit 8 to the analog-to-digital converter 16, there can be a higher output impedance (or resistance) at the voltage divider circuit compared to at the analog-to-digital converter. Thus, the voltage buffer amplifier, positioned between these two circuits or components can act to prevent the analog-to-digital converter from interfering with the voltage divider circuit (where the output or sensed voltage is generated). Stated another way, the analog-to-digital converter can be electrically isolated or buffered with respect to the voltage divider circuit so that the analog-to-digital converter does not load the voltage divider circuit, for example. This can provide for generating a more accurate output voltage measurement that does not receive any appreciable interference from other components downstream from the voltage buffer amplifier.

In one example, if the output voltage, or sensed voltage (Vₛ), is essentially or substantially unchanged as it passes through the voltage buffer amplifier (e.g., the output voltage is transferred unchanged, the voltage gain is 1, or A=1), the voltage buffer amplifier can be referred to as a unity gain buffer, or voltage follower. As a note, even though unity gain buffers are commonly referred to as having a gain that equals 1, in reality they are more accurately referred to as having a voltage gain of approximately 1, as complete unity tends to be theoretical rather than practical. However, the change in voltage can be insignificant enough that they are commonly referred to in the electrical arts as having a voltage gain of 1 (or the equivalent of 0 db). A voltage change of up to 5% can be considered to be unity in accordance with the present disclosure, though often any change may typically be less than 5%. In further detail, however, even though there is little to no voltage gain, unity gain buffers can increase in power due to increase in current, which may be associated with maintaining the voltage at near unity.

Unity gain buffers can be constructed by applying negative feedback, such as is the case with an operational amplifier. With an operational amplifier in the context of the present disclosure, the output voltage, or sensed voltage (Vₛ), can be electrically coupled to a non-inverting input of the operational amplifier, and an output of the operational amplifier can be connected to its inverting input. Thus, the output voltage is fed back into the inverting input, and there is essentially no voltage gain, though there may be an increase in current.

In this example, a difference between the non-inverting input voltage and the inverting input voltage is amplified by the operational amplifier, which can cause the operational amplifier to adjust its output voltage to essentially equal the input voltage. The voltage buffer amplifier shown in FIGS. 1, 2, 4, and 5 at 18 is an example of an operational amplifier.

Typically, the input impedance for an operational amplifier can be relatively high, e.g., 10¹³ Ohm, and thus, the input does not place a load on the source, which in this case is the output voltage generated by the voltage divider circuit 8. This allows the source to draw minimal current (as current is expected to be increased at the operational amplifier. In further detail, the analog-to-digital converter 16 can have a relatively high impedance input, e.g., 1 MOhm to 10 MOhm, so the 1 mA to 1000 mA output current of the operational amplifier can be sufficient to drive the analog-to-digital converter for measurement as a reliable voltage source that mirrors the output or sensed voltage (Vₛ). In some examples, the respective connections to the operational amplifier can be bridging connections, and thus, can provide the benefits of reducing power consumption and electromagnetic interference, e.g., crosstalk, distortion, etc. Many operational amplifiers, or other unity gain buffers, can be provided as a component device or as an integrated circuit (IC), for example, for use with the direct current drive circuitry device of the present disclosure.

In one example, the voltage buffer amplifier 18 can not only provide buffering or isolation between the output voltage, or sensed voltage (Vs), and the analog-to-digital converter 16, but the voltage buffer amplifier can also generate a modest to more significant voltage gain (Vₐ), e.g., voltage gain ranging from about 1 to about 100. In these examples, the direct current drive circuitry device 10 may be more suitable for operation at lower voltages, such as from about 0.1 V to 2 V and then the sensed voltage (Vₛ) can be amplified to increase the output voltage to from about 1 V to about 5V for example. Regardless of whether the voltage buffer amplifier is a unity gain amplifier (A=about 1), or is greater than about 1, the voltage buffer amplifier can still be configured to provide isolation or buffering between the voltage divider circuit 8 and the analog-to-digital converter.

The direct current drive circuitry device 10 can be formed from any substrate used for carrying circuitry, such as silicon, polymer, or other semi-conductive or insulative material. Other materials can likewise be suitable to use with the direct current drive circuitry described herein, in some cases with various layers of different types of materials. Furthermore, the circuitry can be prepared as a printed circuit board in one example, or using any other fabrication technique used for preparing drive circuitry such as that described herein. In addition to the use of printing to prepare the various circuits and traces, the drive circuitry can be prepared, for example, using wire bonding, die bonding, flip chip mounting, surface mount interconnects, etc. Integrated circuits can be used for various components, such as the voltage buffer amplifier 18 and/or the analog-to-digital converter 16.

Turning now to the microfluidic chip 20, as well as the microfluidic sensor 22 associated therewith, there are any of a number of arrangements of sensor circuitry and/or microfluidics that can be used to deliver biological fluids into contact with the microfluidic sensor. Typically, the microfluidic sensor can be a grounded circuit that includes a sensor resistor, such as that shown at sense resistance (Rₛ). Other circuitry components can be present, such as capacitors (C₁), diodes, light-emitting diodes (LEDs), transistors, integrated circuits of various types, etc. The microfluidic sensors can be, for example, a thermal sensor resistor (TSR) that can add heat to a biological fluid in contact with the microfluidic sensor. A TSR can also be considered to be a microfluidic sensor because as a biological fluid is being heated, the temperature of the biological fluid can be “sensed” as a change in resistance resulting from the modified temperature of the biological fluid. In further detail, microfluidic sensors can be used for cell counting applications, blood coagulation applications, identification of the pathogens in nucleic acids, or the like.

In some examples, the microfluidic chip 20 can include multiple microfluidic testing chambers with one or more microfluidic sensors contained or partially contained within respective microfluidic testing chambers. Thus, individual microfluidic sensors 22 can be electrically associated with individual direct current drive circuitry portions (one circuitry portion shown) of the device. On the other hand, there can be examples where multiple microfluidic sensors are driven by common single direct current drive circuitry of the device, or wherein multiple direct current drive circuitry arrangements can be used for a common microfluidic testing chamber, e.g., when there are two different microfluidic sensors in a single microfluidic testing chamber.

In further detail, biological fluids, such as blood or other bodily fluids of humans or other mammals or any other biological fluids, can be tested using the microfluidic sensor 22, and biological fluid information can be ascertained based on electrical properties sensed at the voltage divider circuit 8 and measured at the analog-to-digital converter 16. In certain specific examples and in further detail, various types of biological fluid testing can be used to monitor patients taking various medications, or for diagnostic purposes, or in response to a variety of medical conditions, for example, and can be used as an indicator of patient health. Often, patients may undergo significant clinical testing for various purposes, which can be expensive and inconvenient if performed in a centralized lab. Thus, it can be advantageous to provide an at-home test kit, or a quick test kit for use at a clinic or physician's office (or even at a hospital), that can be relatively inexpensive to use and is user friendly.

Using blood as an example of a specific biological fluid, various forms thereof can be used, e.g., whole blood, diluted blood, blood plasma, platelet-free plasma, platelet-poor plasma, platelet-rich plasma, etc. Likewise, various forms of other types of biological fluids can be used as well. Again, using blood as an example, different aliquots of a common blood sample can be tested, or a plurality of different blood samples can be tested, such as blood samples from different sources, whole blood and plasma samples from a common source, etc. Once the blood sample is loaded into the microfluidic testing chamber, coagulation of the blood sample can be detected in a variety of ways, but in accordance with examples of the present disclosure, the direct current drive circuitry device 10 of the present disclosure can be used to drive the microfluidic chip 20 or microfluidic sensor 22 thereof in some manner, e.g., sensing, heating, both, etc.

In further detail, blood coagulation can be detected thermally. For example, blood can be loaded and flowed through a microfluidic testing chamber, and as it coagulates, the blood sample may generally increase in viscosity, which can detectably change the thermal properties of the blood sample. Thus, a thermal sensor resistor (TSR) can be used to measure the temperature change and correlate the temperature change to coagulation. For example, when a blood coagulation test begins, a voltage can be applied to the TSR across a voltage divider circuit, as previously described, for purposes of measurement. In another example, the blood sample can be pulsed with heat to change the temperature of the blood sample to measure and/or generate a thermal profile, and the change that occurs as coagulation proceeds can be used to determine when clotting occurs. In other examples, a temperature ramp can be initiated when testing begins and an amount of input heat used to maintain the temperature ramp can be monitored as blood coagulation proceeds to generate a thermal profile. This thermal profile can then be used to determine when clotting occurs. Other similar example testing protocols can be used implementing the direct current drive circuitry device of the present disclosure relative to blood coagulation. As a note, the microfluidic sensor 22 used to detect thermal change can be within the microfluidic test chamber, or can be in thermal communication therewith to sense a temperature change from the outside of the chamber. In still further examples, thermal sensing can be used in combination with optical sensing. In other examples, a chemical coagulating agent or anti-coagulating agent can be used with the blood sample in combination with various testing protocols used in conjunction with the direct current drive circuitry device described herein. In other examples, using a sensor to detect the movement of blood cells through the sensor can detect coagulation when that blood cell movement ceases due to a coagulation event occurring.

In one example, the microfluidic chip 20 can include a substrate with circuitry mounted thereon or therein, some of which can be circuitry associated with the microfluidic sensor 22. The microfluidic sensor can be found within a microfluidic testing chamber, for example, which receives a biological fluid to be tested therein. The biological fluid can be loaded conventionally using a dropper, pipette, or other loading device, or can be loaded using microfluidic channels and/or openings. Vents may also be included to allow for a volume of the biological fluid to be tested to displace any air or other fluid that may be present in the microfluidic testing chamber. The microfluidic testing chamber can have any dimension that is appropriate for the volume of biological fluid being tested. For blood samples, where the biological fluid may be more readily available, larger microfluidic testing chamber volumes can be used, e.g., up to about 1 mL or more. With other biological fluids (or even with blood in some examples), there may be benefits to using less biological fluid. That being stated, a reasonable volume of biological fluid that can be tested can be from 1 nL to 1 mL, from 1 nL to 100 μL, from 1 nL to 500 μL, from 100 nL to 500 μL, from 100 nL to 1 μL, from 50 nL to 10 μL, from 1 μL to 1 mL, etc.

Various types of sensors can be used, such as for example a temperature regulator, such as a resistive heater, a peltier heater, or a combination thereof. Thermal sensors can also be used, such as a thermocouple, a thermistor, a thermal sensor resistor, the like, or a combination thereof. In other examples, in addition to the electrical sensor of some type, there can also be an optical sensor associated therewith. Optical sensors can include a photodiode, a phototransistor, a camera with microscope, the like, or a combination thereof.

Any suitable substrate can be used to carry the electronics and/or microfluidic test chamber of the microfluidic chip 20, with the microfluidic sensor 22 included therewith, e.g., outside of the microfluidic test chamber or partially or fully within the microfluidic test chamber. Intervening layers may be used with some of these substrates to provide appropriate semi-conducting, semi-insulative, or dielectric properties, as may be desirable for electronic circuitry, for example. Regardless, the substrate can be prepared from materials such as metal, glass, silicon, silicon dioxide, a ceramic material (e.g. alumina, aluminum borosilicate, etc.), a polymer material (e.g. polyethylene, polypropylene, polycarbonate, poly(methyl methacrylate), epoxy molding compound, polyamide, liquid crystal polymer (LCP), polyphenylene sulfide, polydimethylsiloxane, etc.), and the like, or a combination thereof.

As used herein, the term “about” is used to provide flexibility to a numerical range endpoint by providing that a given value may be “a little above” or “a little below” the endpoint. The degree of flexibility of this term can be dictated by the particular variable and can be determined based on experience and the associated description herein.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, dimensions, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a weight ratio range of about 1 wt % to about 20 wt % should be interpreted to include not only the explicitly recited limits of 1 wt % and about 20 wt %, but also to include individual weights such as 2 wt %, 11 wt %, 14 wt %, and sub-ranges such as 10 wt % to 20 wt %, 5 wt % to 15 wt %, etc.

As a further note, in the present disclosure, it is noted that when discussing the direct current drive circuitry devices or the direct current electrical sensing systems, each of these discussions can be considered applicable to the other examples, whether or not they are explicitly discussed in the context of that example. Thus, for example, in discussing details about the pull-up resistor in the context of the direct current drive circuitry devices, such discussion is also relevant to the various systems described herein, and vice versa.

What is claimed is:

1. A direct current drive circuitry device, comprising:
a pull-up resistor to receive an input voltage;
an electrical interface positioned in series and downstream from the pull-up resistor, wherein the electrical interface is electrically coupleable to a grounded microfluidic sensor to form a voltage divider circuit in combination with the pull-up resistor to generate an output voltage at the voltage divider circuit;
an electrical switch to receive and charge cycle the input voltage to the pull-up resistor of the voltage divider circuit, the charge cycle including a discharging period and a charging period;
an analog-to-digital convertor electrically coupled to the voltage divider circuit to measure the output voltage; and
a voltage buffer amplifier positioned between the voltage divider circuit and the analog-to-digital converter to prevent the analog-to-digital converter from loading the voltage divider circuit.

2. The direct current drive circuitry device of claim 1, wherein the input voltage is from 0.1 V to 5 V and wherein the pull-up resistor has a resistance from 1 KOhm to 2 MOhm.

3. The direct current drive circuitry device of claim 1, wherein the analog-to-digital converter converts the output voltage passed through the voltage buffer amplifier to a digital output value related to a magnitude of the output voltage, and wherein the digital output value has a resolution of 0.02 V or less per digital output value change.

4. The direct current drive circuitry device of claim 1, wherein the voltage buffer amplifier is a unity gain buffer amplifier having a voltage gain of about 1 to deliver the output voltage to the analog-to-digital converter at a voltage level that is about equivalent to output voltage prior to passing through the unity gain buffer amplifier.

5. The direct current drive circuitry device of claim 4, wherein the unity gain buffer amplifier includes an operational amplifier.

6. The direct current drive circuitry device of claim 1, further comprising a rapid discharge pathway to discharge the voltage divider circuit, wherein the rapid discharge pathway includes:
a second electrical switch to be operated oppositionally with respect to the electrical switch, and
a diode connected in parallel with the pull-up resistor to bypass the pull-up resistor and pass through the second electrical switch to ground during discharging periods.

7. A direct current electrical sensing system, comprising:
a voltage source to generate an input voltage;
an electrical switch to charge cycle the input voltage, the charge cycle including a discharging period and a charging period;
a voltage divider circuit to receive the input voltage charge cycled by the electrical switch, wherein the voltage divider circuit generates an output voltage that is lower than the input voltage, the voltage divider circuit, comprising:
a pull-up resistor, and
a microfluidic sensor including a sensor resistor, wherein the
microfluidic sensor is grounded and is connected
downstream and in series with respect to the pull-up resistor;
an analog-to-digital convertor electrically coupled to the voltage divider circuit to measure the output voltage; and
a voltage buffer amplifier positioned between the voltage divider circuit and the analog-to-digital circuit to prevent the analog-to-digital converter from loading the voltage divider circuit,
wherein the direct current electrical sensing system includes a direct current drive circuitry device which carries the electrical switch, the pull-up resistor, the analog-to-digital convertor, and the voltage buffer amplifier.

8. The direct current electrical sensing system of claim 7, wherein the analog-to-digital converter converts the output voltage to a digital output value related to a magnitude of the output voltage, and wherein the digital output value has a resolution of 0.02 V or less per digital output value change.

9. The direct current electrical sensing system of claim 7, wherein the voltage buffer amplifier is a unity gain buffer amplifier having a voltage gain of about 1 to deliver the output voltage to the analog-to-digital converter at a voltage level that is about equivalent to output voltage prior to passing through the unity gain buffer amplifier.

10. The direct current electrical sensing system of claim 7, wherein the voltage buffer amplifier generates a voltage gain of greater than about 1 to about 100 to deliver the output voltage to the analog-to-digital converter at a voltage level that is greater than the output voltage prior to passing through the voltage buffer amplifier.

11. The direct current electrical sensing system of claim 10, wherein the input voltage is from 0.1 V to 2 V.

12. The direct current electrical sensing system of claim 7, wherein the sensor resistor has a resistance within one order of magnitude of the pull-up resistor.

13. The direct current electrical sensing system of claim 7, further comprising a rapid discharge pathway to discharge the voltage divider circuit, wherein the rapid discharge pathway includes:
a second electrical switch to be operated oppositionally with respect to the electrical switch, and
a diode connected in parallel with the pull-up resistor to bypass the pull-up resistor and pass through the second electrical switch to ground during discharging periods.

14. A direct current electrical sensing system, comprising:
a voltage source to generate an input voltage ranging from 0.1 V to 5 V;
an electrical switch to charge cycle the input voltage, the charge cycle including a discharging period and a charging period;
a voltage divider circuit to receive the input voltage charge cycled by the electrical switch, wherein one charge cycle includes one discharging period and one charging period at the voltage divider circuit, and wherein the voltage divider circuit generates an output voltage that is lower than the input voltage, the voltage divider circuit, comprising:
a pull-up resistor having a resistance from 1 KOhm to 2 MOhm, and
a microfluidic sensor including a sensor resistor, wherein the
microfluidic sensor is grounded and is connected
downstream and in series with respect to the pull-up resistor;
an analog-to-digital convertor electrically coupled to the voltage divider circuit to measure the output voltage; and
a voltage buffer amplifier positioned between the voltage divider circuit and the analog-to-digital convertor to prevent the analog-to-digital convertor from loading the voltage divider circuit,
wherein the output voltage is measurable at the analog-to-digital converter upon charging the voltage divider circuit during a sampling cycle at a sampling cycle rate from 1 microseconds to 1000 microseconds, wherein the direct current electrical sensing system includes a direct current drive circuitry device which carries the electrical switch, the pull-up resistor, the analog-to-digital convertor, and the voltage buffer amplifier.

15. The direct current electrical sensing system of claim 14, wherein the microfluidic sensor is in contact with:
a diluted blood sample for a cell counting application, wherein the sampling cycle rate is from 10 microseconds to 100 microseconds;
a blood sample for causing coagulation, wherein the sampling cycle rate is from 100 microseconds to 350 microseconds; or a nucleic acid sample for sample identification, wherein the sampling cycle rate is from 25 microseconds to 1000 milliseconds.
